(19) United States
(12) Patent Application Publication
(10) Pub. No.: US 2017/0224253 A1
(43) Pub. Date: Aug. 10, 2017

(54) PATIENT BED-EXIT PREDICTION AND DETECTION

(71) Applicant: Covidien LP, Mansfield, MA (US)

(72) Inventors: DAVID BENJAMIN BERLIN, Niwot, CO (US); BETH A. ANDREWS, Altadena, CA (US)

(21) Appl. No.: 15/040,733

(22) Filed: Feb. 10, 2016

(51) Int. Cl. A61B 5/11; G08B 21/04

(52) U.S. Cl. CPC A61B 5/1115; G08B 21/0446

(57) ABSTRACT

Methods, systems, and devices for predicting bed-exit of a patient are described. Methods may include receiving pressure data from a pressure sensor disposed under the patient, receiving accelerometry data from an accelerometer coupled with the patient, detecting movement of the patient based at least in part on the pressure data and the accelerometry data, and determining whether the detected movement is indicative of bed-exit based at least in part on the detected movement. Systems may include a pressure sensor configured to collect pressure data associated with movement of the patient, an accelerometer configured to collect accelerometry data associated with movement of the patient, and a central aggregator configured to determine whether the movement of the patient is indicative of bed-exit based at least in part on the collected pressure data and the collected accelerometry data.

### BACKGROUND

The following relates generally to wireless patient monitoring systems and medical sensors, and more specifically to patient bed-exit prediction and detection.

In a medical care facility such as a hospital, a patient may spend a significant amount of time on a medical bed, for example when recovering from surgery or during treatment for an affliction. On occasion, the patient may attempt to leave the bed (e.g., to use the restroom or perform some task). However, the patient may not be in condition to exit the bed (e.g., the patient may be too weak, medicated, or injured to successfully complete a bed-exit maneuver). In such cases, if assistance is not available, the patient may fall and sustain injury.

Some hospitals may use monitoring techniques that attempt to alert a remote clinician when a patient is leaving his bed. The alerted clinician may travel to the patient's location to assist with, or stop, the patient exiting the bed. But conventional monitoring techniques may be hyper-sensitive or insensitive. A hyper-sensitive alert system may erroneously detect patient bed-exit and issue false alarms, which can lead to alarm fatigue or pull a clinician away from other duties. An insensitive alert system may fail to detect, or belatedly detect, patient bed-exit, which may result in the patient risking harm by getting out of bed unassisted. Thus, errors in bed-exit detection techniques may needlessly alert clinicians or increase the frequency of unassisted patient bed-exit.

### SUMMARY

The described features generally relate to methods and devices for detecting bed-exit of a patient to reduce or eliminate the risk of the patient attempting to exit their bed unassisted. Patient movement may be detected that is indicative of an intent to exit a bed. The movement may be detected via analysis of data from a pressure sensor and an accelerometer coupled with the patient. The detected movement may be compared to patterns of movement that are known to be indicative of bed-exit attempts. If the detected movement resembles a pattern of movement that is indicative of a bed-exit attempt, an alarm may be triggered that alerts a clinician of the patient's intent to exit the bed.

In certain situations, a patient disposed on a medical bed may attempt to leave, or exit, the medical bed. In such cases, to mitigate the risk of the patient falling while exiting the bed, an apparatus may determine that movement of the patient is indicative of bed-exit and trigger an alarm. In accordance with methods described herein, the movement may be detected from analysis of data from a pressure sensor and an accelerometer.

Embodiments of systems and devices for predicting and detecting patient bed-exit are also described. In accordance with certain aspects, a system includes a pressure sensor configured to collect pressure data associated with movement of a patient, an accelerometer configured to collect accelerometry data associated with movement of the patient, and a central aggregator configured to determine whether the movement of the patient is indicative of bed-exit.

Certain embodiments of the present disclosure may include some, all, or none of the above advantages or features. One or more other technical advantages or features may be readily apparent to those skilled in the art from the figures, descriptions, and claims included herein. Moreover, while specific advantages or features have been enumerated above, various embodiments may include all, some, or none of the enumerated advantages or features.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

FIG. 1 illustrates an example of a wireless sensor system that supports patient bed-exit prediction and detection in accordance with aspects of the present disclosure;

FIG. 2 illustrates an example of a wireless sensor system that supports patient bed-exit prediction and detection in accordance with aspects of the present disclosure;

FIG. 3 illustrates an example of a process flow in a system that supports patient bed-exit prediction and detection in accordance with aspects of the present disclosure;

FIG. 4 shows a block diagram of a wireless device that supports patient bed-exit prediction and detection in accordance with aspects of the present disclosure;

FIG. 5 shows a block diagram of a wireless device that supports patient bed-exit prediction and detection in accordance with aspects of the present disclosure;

FIG. 6 shows a block diagram of a wireless device that supports patient bed-exit prediction and detection in accordance with aspects of the present disclosure;

FIG. 7 illustrates a method for patient bed-exit prediction and detection in accordance with aspects of the present disclosure;

FIG. 8 illustrates a method for patient bed-exit prediction and detection in accordance with aspects of the present disclosure; and

FIG. 9 illustrates a method for patient bed-exit prediction and detection in accordance with aspects of the present disclosure.

### DETAILED DESCRIPTION

In accordance with various embodiments described herein, patient bed-exit may be detected or predicted using data from multiple sensors. The data may be associated with movement of the patient as well as physiological parameters or conditions of the patient (e.g., vital signs or disease states). For example, the data may be from a pressure sensor disposed beneath the patient and an accelerometer physically coupled with the patient. The movement of the patient, as indicated by the two different sensors, may be used to determine whether the patient is attempting to exit the bed.

A pressure sensor (e.g., a bed pressure mat) may be disposed under a patient lying on a medical bed (e.g., the pressure sensor may be placed on or integrated with a mattress of the bed). The pressure sensor may sense pressure exerted by the patient on the pressure sensor and use this information to detect movement by the patient. For example, pressure experienced by the pressure sensor when the patient is lying down may be different than pressure experienced when the patient is attempting to get out of bed (e.g., when the patient sits up, shifts, repositions one or more limbs, rolls over, etc.). Thus, data from the pressure sensor may be used to detect movement of a patient that is indicative of patient bed-exit. The pressure sensor data may be used in conjunction with other data (e.g., accelerometry data) to predict and detect patient bed-exit.

An accelerometer may be coupled with the patient (e.g., coupled with the chest of the patient) and collect data associated with movement of the patient. The accelerometer may determine when a patient is moving by sensing acceleration associated with the movement. For example, an accelerometer coupled with the chest of a patient may sense movement of the patient when the patient sits up from a supine position. Data from the accelerometer may be used in conjunction with other data (e.g., pressure sensor data) to predict and detect a patient's attempt to leave a medical bed. In some cases, a bed-exit attempt detected by the accelerometry data is verified by cross-checking it with pressure sensor data. In other cases, the accelerometry data may be used to verify a bed-exit attempt identified using pressure sensor data. In accordance with various embodiments described herein, the accelerometry data and pressure sensor data may be used to predict bed-exit attempts before they occur.

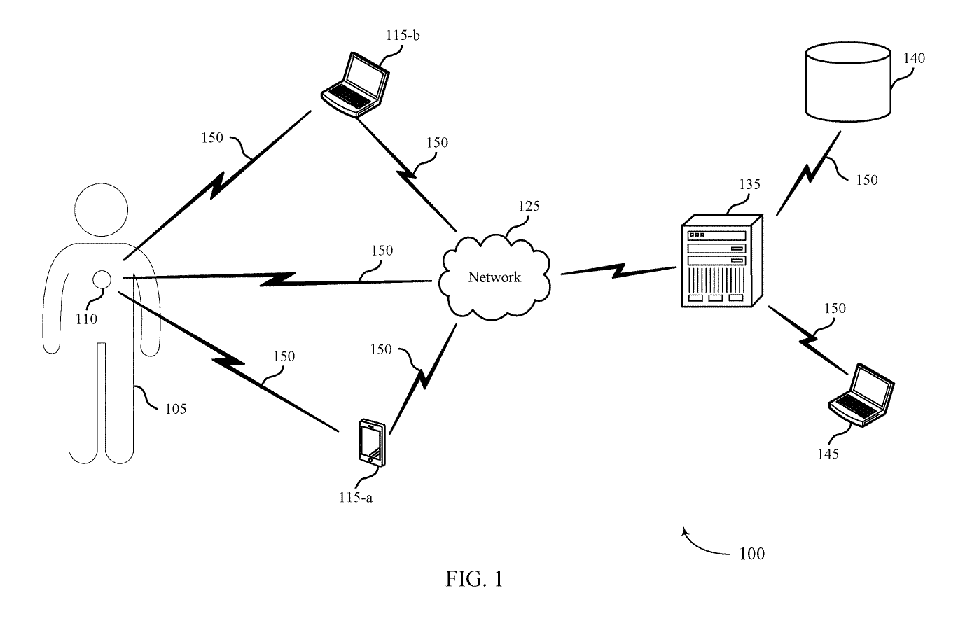

FIG. 1 illustrates an example of a wireless patient monitoring system 100 in accordance with various embodiments of the present disclosure. The wireless patient monitoring system 100 includes a patient 105 wearing, carrying, disposed on, or otherwise coupled with a sensor unit 110. Although a single sensor unit 110 is shown, multiple sensor units 110 may be worn by the patient 105. The patient 105 may be a patient in a hospital, nursing home, home care, or other medical care facility. The sensor unit 110 may transmit signals via wireless communications links 150 to local computing devices 115 or to a network 125. In some cases, the sensor unit 110 may be used in conjunction with another sensor unit 110 to detect movement of the patient. The movement may be indicative of an attempt of the patient 105 to perform some particular activity (e.g., bed-exit).

Local computing device 115-a may be a wireless device such as a tablet, cellular phone, personal digital assistant (PDA), dedicated receiver or other similar device or a spatially distributed network of devices configured to receive signals from the sensor unit 110. Local computing device 115-b may be a wireless laptop computer or mobile computer station also configured to receive signals from the sensor unit 110. In accordance with various embodiments, a local computing device 115 may be configured to receive data associated with movement of the patient 105. In some embodiments, the data may be used to identify patterns of movement that indicate an attempt by the patient 105 to exit a bed upon which the patient 105 is disposed. The local computing devices 115 may be in communication with a central station 135 via network 125.

The sensor unit 110 may also communicate directly with the central station 135 via the network 125. The central station 135 may be a server or a nurses station located within the hospital or in a remote location. The central station 135 may be in further communication with one or more remote computing devices 145, thus allowing a clinician to remotely monitor the patient 105. The central station 135 may also be in communication with various remote databases 140 where the collected data may be stored.

The sensor unit 110 may include one or more sensors configured to collect a variety of physiological parameters as well as information related to the location and movement of the patient 105. For example, the sensor unit 110 may include a pulse oximetry (SpO2) sensor, a heart rate sensor, a blood pressure sensor, a pressure sensor, an electrocardiogram (ECG) sensor, a respiratory rate sensor, a glucose level sensor, a body temperature sensor, an accelerometer, a global positioning sensor, a sensor which triangulates position from multiple local computing devices 115, and any other sensor configured to collect physiological, location, or motion data.

The sensor unit 110 may be coupled with the patient 105 in a variety of ways depending on the data being collected. For example, the sensor unit 110 may be directly coupled with the patient 105 (e.g., physically connected to the patient's chest, worn around the patient's wrist, or attached to the patient's finger). The sensor may be indirectly coupled with the user so that movement of the patient 105 is detected even though the sensor is not in direct contact with, or physically connected to, the patient 105 (e.g., the sensor unit 110 may be disposed under the patient 105). The data collected by the sensor unit 110 may be wirelessly conveyed to either the local computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

In accordance with various embodiments, methods and apparatuses are described for detecting bed-exit of a patient 105 via data from one or more sensor units 110. As described in detail below, detection may include receiving, at a local computing device 115 (or sensor unit 110) data from the sensor unit 110 that indicates movement of a patient 105, and determining that the movement corresponds to a bed-exit attempt. In accordance with various embodiments, the data may be used separately or in conjunction to detect the bed-exit attempt. In some cases, the data may be predictive of a future bed-exit attempt (e.g., more than 1 minute after the movement associated with the data). For instance, the data may be used to determine the likelihood of a bed-exit attempt before it occurs.

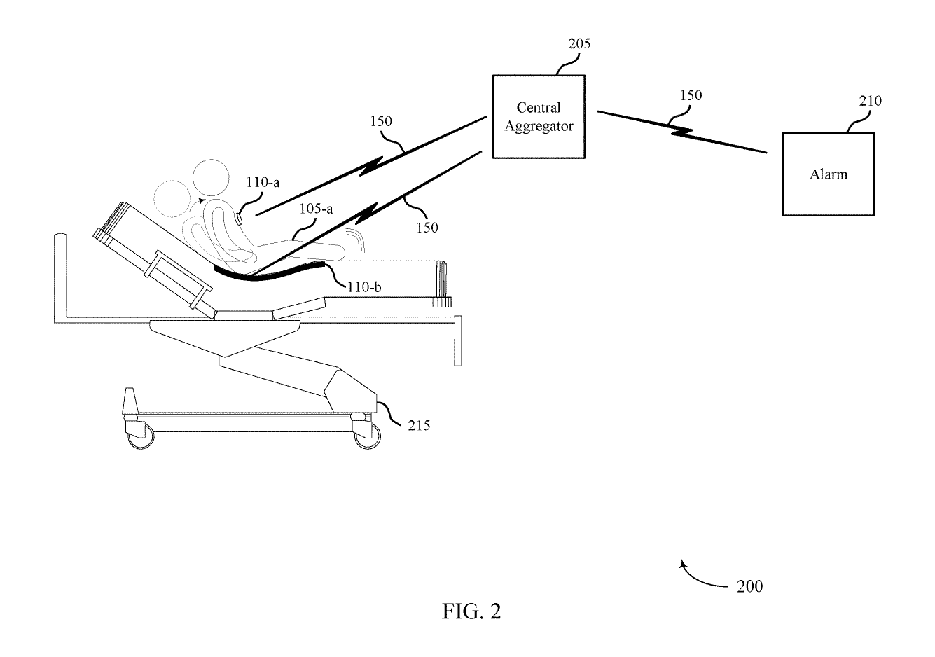

FIG. 2 illustrates an example of a wireless patient monitoring system 200 for patient bed-exit prediction and detection. The wireless patient monitoring system 200 includes an accelerometer 110-a and a pressure sensor 110-b, which may be examples of sensor unit 110 described with reference to FIG. 1. Although only one accelerometer 110-a and one pressure sensor 110-b are shown, the techniques described herein may be implemented using any number of accelerometers and pressure sensors. Accelerometer 110-a and pressure sensor 110-b may communicate with central aggregator 205 via communication links 150. Central aggregator 205 may be a local computing device 115, a central station 135, or a remote computing device 145 as described with reference to FIG. 1. In some examples, the operations of central aggregator 205 may be implemented by a sensor unit 110 (e.g., by accelerometer 110-a) or by the medical bed 215. In some cases, wireless patient monitoring system 200 includes an alarm 210. Alarm 210 may be part of an alarm system that is configured to provide an indication of bed-exit (e.g., to a clinician).

Patient 105-a may be resting on medical bed 215, which may be situated in a health care facility or environment (e.g., in a hospital, clinic, assisted living community, personal residence, etc.). While lying in medical bed 215, patient 105-a may be in various positions (e.g., patient 105-a may be prone, supine, or on a side). At some point, patient 105-a may attempt to leave the medical bed 215. To do so, patient 105-a may perform a pattern of movement to assume a number of new positions that lead to bed-exit (e.g., patient 105-a may sit up and swing their legs over the side of the medical bed 215). In accordance with various embodiments described herein, the movement of patient 105-a may be detected by accelerometer 110-a and pressure sensor may 110-b and used to identify a bed-exit attempt of patient 105-a. In some cases, the alarm 210 may be triggered in response to the bed-exit identification, which may alert a clinician of the bed-exit attempt.

As disclosed herein, accelerometer 110-a and pressure sensor 110-b may collect data associated with movement of patient 105-a. For example, the weight of patient 105-a may apply pressure to pressure sensor 110-b, which may be disposed under patient 105-a (e.g., pressure sensor 110-b may be a bed mat pressure sensor). When patient 105-a moves, the pressure sensed by pressure sensor 110-b may change; thus, changes in pressure may be associated with movement of patient 105-a. For instance, the pressure experienced by pressure sensor 110-b may increase when patient 105-a sits up from a lying position due to the decreased area supporting the weight of patient 105-a (e.g., the area may decrease from the combined area of the back and posterior of patient 105-a to the area of just the posterior of patient 105-a). In some cases, the pressure experienced by pressure sensor 110-b may decrease when patient 105-a lifts his body to get closer to the side of the medical bed 215 due to a portion of the weight of patient 105-a being supported by an external object (e.g., a bed railing) rather than by pressure sensor 110-b. Thus, movement of patient 105-a may be represented by data collected by pressure sensor 110-b. Pressure sensor 110-b may use the collected data to detect movement of patient 105-a independently, or pressure sensor 110-b may send the collected data to another device (e.g., central aggregator 205) for analysis.

Movement of patient 105-a may also be detected by analyzing data collected by accelerometer 110-a. Accelerometer 110-a may be coupled (e.g., physically coupled) with patient 105-a so that accelerometer 110-a moves when patient 105-a moves. For example, accelerometer 110-a may be coupled with the chest of patient 105-a so that when the chest of patient 105-a accelerates (e.g., when patient 105-a sits up), accelerometer 110-a experiences acceleration. Accelerometer 110-a may not experience acceleration when patient 105-a is still. Thus, accelerometry data from accelerometer 110-a may indicate movement of patient 105-a. Accelerometer 110-a may use the collected accelerometry data to detect movement of patient 105-a independently, or accelerometer 110-a may send the collected data to another device (e.g., central aggregator 205) for analysis. In some cases, more than one accelerometer 110-a may be coupled with patient 105-a. In such cases, the movement of multiple parts of patient 105-a may be used to detect movement by patient 105-a.

Different movements of patient 105-a may correspond to different data collected by accelerometer 110-a and pressure sensor 110-b. For example, data collected by accelerometer 110-a and pressure sensor 110-b when a patient 105-a shifts may be different from data collected when patient 105-a rolls over. That is, shifting may be associated with a first data set (which includes pressure data or accelerometry data) and rolling over may be associated with a second data set (which includes pressure data or accelerometry data). Thus, data from accelerometer 110-a and pressure sensor 110-b may be used (e.g., by central aggregator 205) to detect various types of movement by patient 105-a. In some cases, the data collected by accelerometer 110-a and pressure sensor 110-b may be combined to detect a type of movement that is undetectable using data from a single sensor (e.g., using only one of accelerometer 110-a and pressure sensor 110-b). For example, data collected by accelerometer 110-a associated with a partial sit-up movement and data collected by pressure sensor 110-b associated with rolling over may be combined to detect a particular movement (e.g., a rotational upper-body lift performed by patient 105-a) that is unrecognizable using data from either sensor 110 independently.

The data collected by accelerometer 110-a and pressure sensor 110-b may be sent to central aggregator 205 (e.g., via communication links 150). The data may be sent using wireless or wired means. The data may be sent automatically (e.g., periodically) or upon a trigger. Data sent from pressure sensor 110-b may be sent independent of data from accelerometer 110-a, and vice versa (e.g., data may be sent from pressure sensor 110-b and accelerometer 110-a synchronously or asynchronously). Central aggregator 205 may receive the data and detect movement of patient 105-a based at least in part on the pressure data and accelerometry data. For example, central aggregator 205 may process and analyze the data to detect and identify movement of patient 105-a as described above. The movement of patient 105-a may be compared to predetermined patterns of movement that are known to be indicative of bed-exit. If the detected movement of patient 105-a matches a predetermined pattern of movement indicative of bed-exit, the central aggregator 205 may determine that patient 105-a is attempting to leave medical bed 215, or will attempt to leave in the future. If the detected movement of patient 105-a does not match a predetermined pattern of movement indicative of bed-exit, the central aggregator 205 may determine that patient 105-a does not intend to leave medical bed 215.

The predetermined patterns of movement that are indicative of bed-exit may be pre-determined (e.g., calibrated or pre-configured) and may be specific to patient 105-a. For example, bed-exit attempt by patient 105-a may be associated with three patterns of movement:

pattern A, pattern B, and pattern C. However, bed-exit of another patient 105 may be associated with three different patterns of movement: pattern D, pattern E, and pattern F. That is, patterns A, B, and C may not be indicative of bed-exit for the other patient. Thus, a central aggregator 205 may be calibrated on a per-patient basis to determine which patterns of movement are indicative of a bed-exit attempt for that particular patient. In some cases, the predetermined patterns of movement that are indicative of bed-exit may be pre-configured for all patients in general (e.g., the predetermined patterns may be movements common among patients that are generally associated with bed-exit).

In some cases, the movement detected by central aggregator 205 may not match a predetermined pattern of movement exactly but may be similar. In these or other cases, central aggregator 205 may determine the extent of the similarity between the two movements (detected and predetermined) and derive a likelihood that the detected movement corresponds to bed-exit. The greater the similarity between the two movements, the greater likelihood of bed-exit the central aggregator 205 may assign to the detected movement. The likelihood of bed-exit may be reflected in the level of alarm output by alarm 210. For instance, a first alarm level may correspond to high likelihood of bed-exit and a second alarm level may correspond to a low likelihood of bed-exit. Thus, a clinician may adjust their reaction to the alarm based on the level of alarm.

Central aggregator 205 may determine movement that is indicative of bed-exit via a weighted algorithm. The data collected by accelerometer 110-a and pressure sensor 110-b may be inputs to the weighted algorithm. In some cases, other factors may serve as inputs to the weighted algorithm, such a vital sign readings or information collected by other sensor units 110. The weighted algorithm may be specific to patient 105-a or may be calibrated for patients in general. According to various embodiments, the weighted algorithm may apply different weights to different parameters based on one or more conditions or traits of patient 105-a, or based on a bed-exit history of patient 105-a. Examples of conditions or traits of a patient include the height and weight of the patient, the family history of the patient, the current and past medical condition of the patient (e.g., the disease state of the patient), the medication of the patient, the physical state of the patient, and various physiological parameters of the patient (e.g., blood pressure, heart rate, respiratory rate, etc.). The bed-exit history of the patient may include the frequency of bed-exit attempts by the patient, the time of day of previous bed-exit attempts, bed-exit fall history, and the elapsed time since the most recent bed-exit attempt.

Other factors may also serve as inputs to the weighted algorithm, including risk levels associated with the patient (e.g., as decided by a clinician), a disease state of the patient, or vital sign readings of the patient (e.g., heart rate and respiratory rate). For example, a patient recovering from hip-replacement surgery may be assigned a higher risk level than a patient recovering from wrist surgery. In another example, the weighted algorithm may be adjusted according to specific a diseases state or condition of the patient (e.g., there may be an Alzheimer-specific algorithm). In some cases, the weighted algorithm may be dynamic (e.g., the weighted algorithm may be updated based on changes in inputs or the environment). In accordance with various embodiments described herein, central aggregator 205 may use a different weighted algorithm based on the time of day, or one of the parameters previously described.

The patterns of movement detected by central aggregator 205 may be indicative of bed-exit attempts within various thresholds of time. For instance, a movement of patient 105-a may indicate a current bed-exit attempt or a predict a future bed-exit attempt before it occurs. In a bed-exit attempt that is classified as a current bed-exit attempt, the movement of the patient which indicates the bed-exit attempt may also be the movement the patient performs to get out of bed. In some examples, a bed-exit attempt that is classified as a current bed-exit attempt may occur within a minute of the detected movement. A bed-exit attempt that is classified as a future bed-exit attempt may be a bed-exit attempt that is predicted before it occurs. For instance, a future bed-exit attempt may be predicted by correlating detected movement of patient 105-a with behavior of patient 105-a that precedes a bed-exit attempt. For example, patient behavior may follow a certain pattern prior to attempting a bed-exit. For instance, patient 105-a may begin shifting or moving their appendages leading up to an attempted bed-exit. Thus, the movements used by a patient to perform a predicted bed-exit attempt may exclude the movements used to predict the bed-exit. In some cases, a bed-exit attempt may be both predicted and detected. For instance, a bed-exit attempt may be predicted by determining that movement of patient 105-a is indicative of an upcoming bed-exit, and then the bed-exit attempt may be detected when movement of patient 105-a is determined to correspond to a live bed-exit attempt.

In some cases, a predictive bed-exit weighted algorithm may be used to alert a clinician when a patient 105-a is more likely to attempt a bed-exit during the night (e.g., to use the restroom). The weighted algorithm may use data from an accelerometer 110-a and pressure sensor 110-b, along with vital sign data, to predict waking and a bed-exit attempt by patient 105-a. For example, the weighted algorithm may monitor vital sign data (e.g., the heart rate or respiratory rate) of patient 105-a and use this information to corroborate a future bed-exit attempt that is predicted using accelerometry data and pressure data. If the vital sign data contradicts the bed-exit prediction (e.g., the vital sign data indicates that the patient is in a deep sleep), the central aggregator 205 may dismiss or ignore the predicted bed-exit. If the vital sign data corroborates the bed-exit predication (e.g., the vita sign data indicates that the patient is on the verge of waking up), the central aggregator 205 may trigger an alarm system (e.g., an alarm system that includes alarm 210) that would indicate to a clinician that the patient 105-a is waking up and may need assistance to use the restroom.

As described above, central aggregator 205 may be calibrated to patient 105-a to determine whether a movement of patient 105-a is indicative of a bed-exit. In some cases, central aggregator 205 may be calibrated to patient 105-a so that patterns of movement of patient 105-a are identified as being indicative of the type of bed-exit (e.g., current or future). These patterns of movement may be dynamically updated based on the bed-exit history of patient 105-a. For example, patient 105-a may have five different methods of exiting the medical bed 215, each of which corresponds to a sequence or pattern of movements. Patient 105-a may also display two different types of behavior prior to attempting bed-exit, each of which corresponds to a particular pattern of movement. Thus, central aggregator 205 may compare movement detected from patient 105-a to the calibrated, predetermined patterns of movement for patient 105-a to determine if the detected movement is indicative of a current bed-exit attempt or a future bed-exit attempt.

In some cases, the movement data from one of the sensor units 110 may be used independent from data from the other sensor unit 110 to detect or predict a bed-exit attempt. In such cases, data from the other sensor unit 110 may be used to verify the detection or predication. For example, central aggregator 205 may detect a pattern of movement using data from pressure sensor 110-b that indicates a bed-exit attempt by patient 105-a. Central aggregator 205 may verify that the pattern of movement is indicative of bed-exit by cross-checking the pattern with data from accelerometer 110-a. If the pattern of movement is verified, central aggregator 205 may determine that the movement is indicative of bed-exit. If the movement is not verified, central aggregator 205 may determine that the movement is not indicative of bed-exit. In some cases, a pattern of movement detected using data from accelerometer 110-a may be verified using data from pressure sensor 110-b. This verification process may occur prior to triggering alarm 210.

The alarm 210 may initiate an alert that indicates to a clinician when a patient is attempting bed-exit. The alert may be in response to a communication from central aggregator 205, which may be conveyed using wired or wireless means (e.g., using wireless communication link 150). In some cases, the sensitivity of the alarm 210 may be adjusted based on various factors associated with patient 105-a (e.g., based on the condition of patient 105-a, the medication level of patient 105-a, or a previous fall history of patient 105-a). For example, the sensitivity of alarm 210 may be increased so that it is more easily triggered when patient 105-a is at greater risk for a fall (e.g., when patient 105-a is heavily medicated). The sensitivity of alarm 210 may be decreased (e.g., when patient 105-a is un-medicated) so that alarm 210 is harder to trigger when patient 105-a is at less risk for a fall.

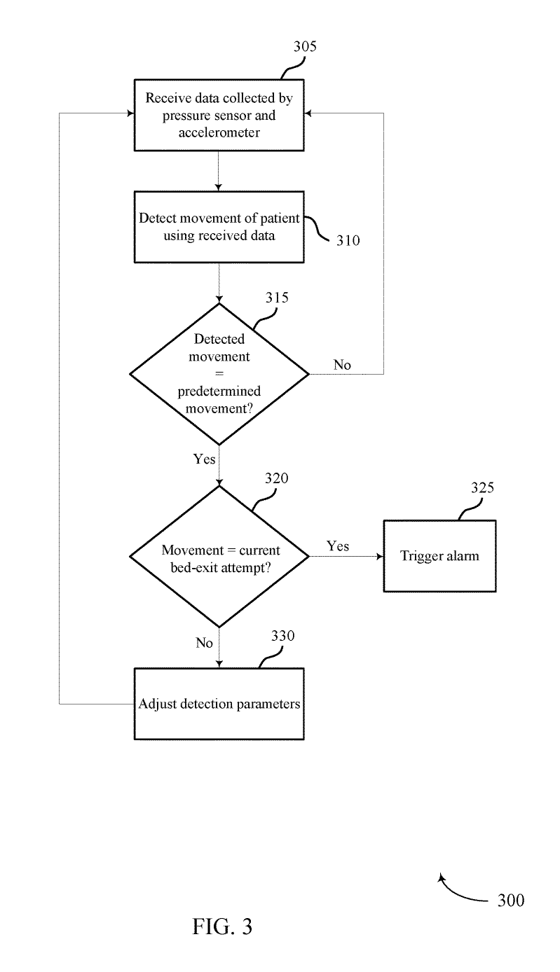

FIG. 3 illustrates an example of a process flow 300 for patient bed-exit prediction and detection in accordance with various aspects of the present disclosure. Process flow 300 may represent operations that are performed by a central aggregator 205 as described with reference to FIG. 2. For example a central aggregator may perform the operations to detect a bed-exit attempt by a patient. The central aggregator may be operable to communicate (e.g., wirelessly or via wired means) with a pressure sensor and an accelerometer. The pressure sensor 110 may be disposed under the patient and may be an example of a pressure sensor 110-b described with reference to FIG. 2. The accelerometer 110-a may be coupled with the patient and may be an example of an accelerometer 110-a described with reference to FIG. 2.

At 305, the central aggregator may receive data collected by the pressure sensor and data collected by the accelerometer. The data may be received at the same or different times, upon request or automatically. The data may be associated with movement of the patient. At 310, the central aggregator may detect movement of the patient based at least in part on the received data. In some cases, the central aggregator may use the received data as inputs to a weighted algorithm that is used to determine the detected movement. The weighted algorithm may be specific to the patient, and may be dynamically updated. In some examples, the detected movement may be compared to a number of predetermined movements (e.g., a calibrated movement). The predetermined movements may be specific to the patient.

At 315, the central aggregator may determine whether the detected movement matches one of the predetermined movements. If the detected movement does not match a predetermined movement, the central aggregator may return to 305 and receive new data from the pressure sensor and accelerometer. If the detected movement matches a predetermined movement, the central aggregator may, at 320, determine the type of bed-exit of which the detected movement is indicative. For example, the central aggregator may determine whether the movement is indicative of a current bed-exit attempt or a future bed-exit attempt.

If the movement is indicative of a current bed-exit attempt (e.g., a bed-exit within one minute), the central aggregator may trigger an alarm at 325. The central aggregator may trigger the alarm by sending an indication (e.g., via wireless or wireless means) of the bed-exit to the alarm. The alarm may alert a clinician of the imminent bed-exit. If the movement is not indicative of a current bed-exit attempt, the central aggregator may determine that the movement is indicative of a future bed-exit. Accordingly, at 330, the central aggregator may adjust detection parameters such as the weighted algorithm or frequency with which the sensors (e.g., the accelerometer or pressure sensor) report data to the central aggregator. In some cases, the central aggregator may also communicate with the alarm. For example, the central aggregator may indicate the future bed-exit to the alarm. The alarm may alert a clinician that a future bed-exit is expected.

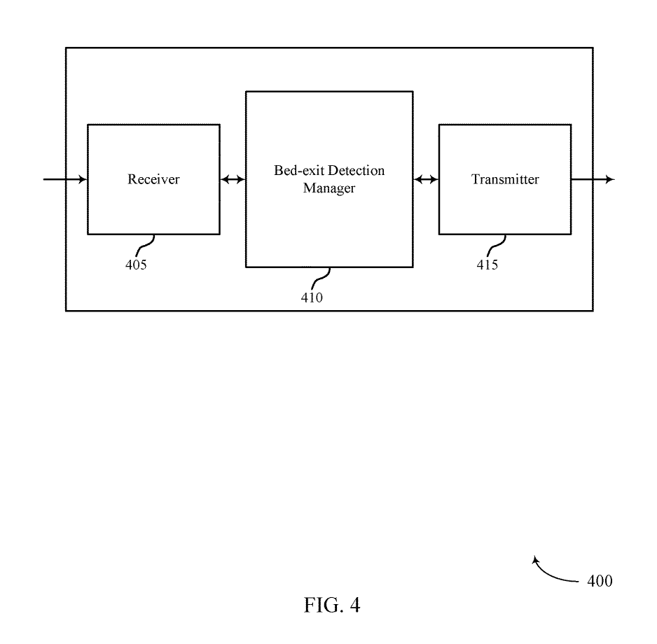

FIG. 4 shows a block diagram of a wireless device 400 that supports patient bed-exit prediction and detection in accordance with various aspects of the present disclosure. The wireless device 400 may communicate via wired or wireless means and may be an example of aspects of a central aggregator 205 described with reference to FIGS. 2 and 3. Wireless device 400 may include receiver 405, bed-exit detection manager 410 and transmitter 415. Wireless device 400 may also include a processor. Each of these components may be in communication with each other. Wireless device 400 may be operable to detect movement of the patient based at least in part on data associated with movement of the patient (e.g., pressure data and accelerometry data) and detect movement of the patient based at least in part on pressure data and accelerometry data associated with movement of the patient.

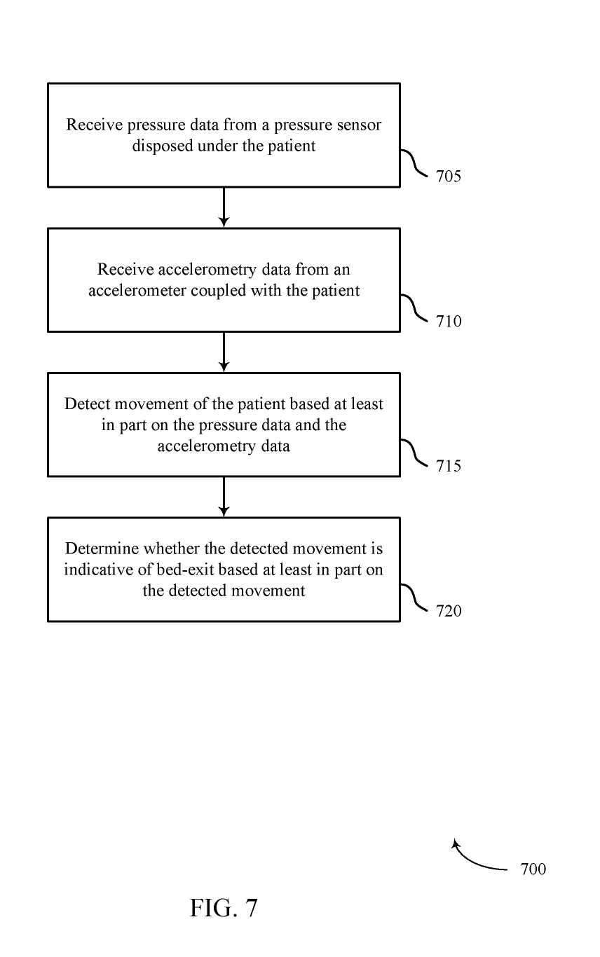

The receiver 405 may receive information such as packets, user data, vital sign data, or control information associated with various sensor units (e.g., pressure sensors or accelerometers). For example, the receiver 405 may receive pressure data associated with movement of a patient from a pressure sensor disposed under the patient such as described with reference to FIGS. 2 and 3. The receiver 405 may also receive accelerometry data associated with movement of a patient from an accelerometer coupled with the patient. The receiver 405 may receive data via wireless or wired means (e.g., the wireless device 400 may receive the pressure data and the accelerometry data wirelessly). The receiver 405 may pass data and information on to other components (e.g., to the bed-exit detection manager 410) of the wireless device 400. The receiver 405 may be an example of aspects of the transceiver 625 described with reference to FIG. 7.

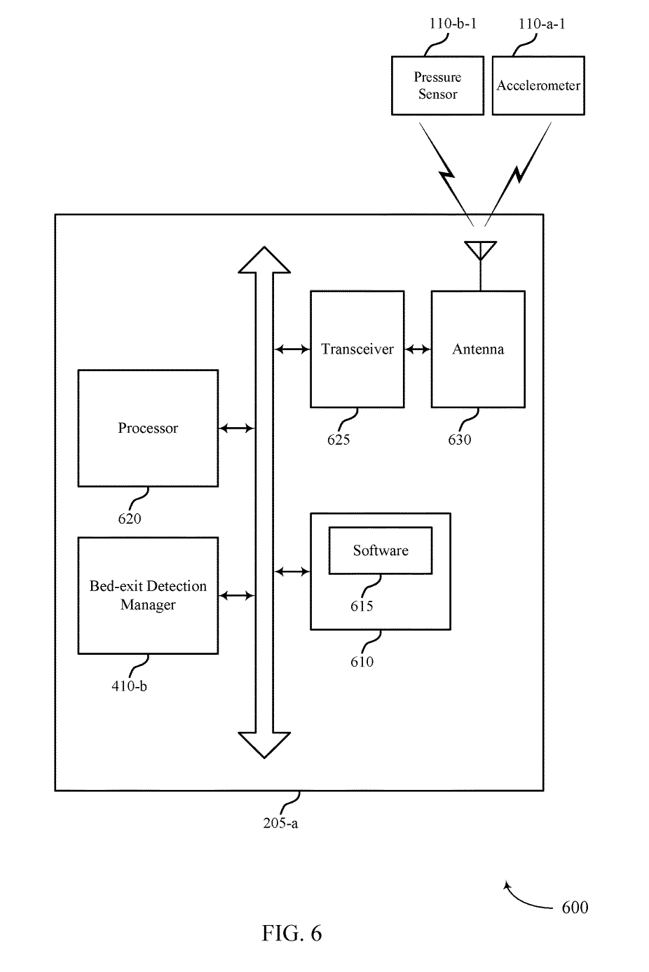

The bed-exit detection manager 410 may include circuitry, logic, hardware or software for collecting and processing the data received from one or more sensors. For example, the bed-exit detection manager 410 may process and analyze (e.g., using a weighted algorithm) pressure data and accelerometry data from a pressure sensor and an accelerometer, respectively. The bed-exit detection manager 410 may detect movement of a patient based on the pressure data and the accelerometry data and determine whether the detected movement is indicative of bed-exit. The determination may be based at least in part on the detected movement. The bed-exit detection manager 410 may be an example of aspects of the bed-exit detection manager 410-b described with reference to FIG. 6.

The transmitter 415 may transmit signals received from other components of the wireless device 400. In some examples, the transmitter 415 may be collocated with a receiver in a transceiver module. For example, the transmitter 415 may be an example of aspects of the transceiver 625 described with reference to FIG. 7. The transmitter 415 may include a single antenna, or it may include multiple antennas. In some cases, the transmitter 415 may transmit a signal to an alarm that is indicative of patient bed-exit. The signal may be in response to detection of a bed-exit attempt (e.g., a current bed-exit attempt or a future bed-exit attempt).

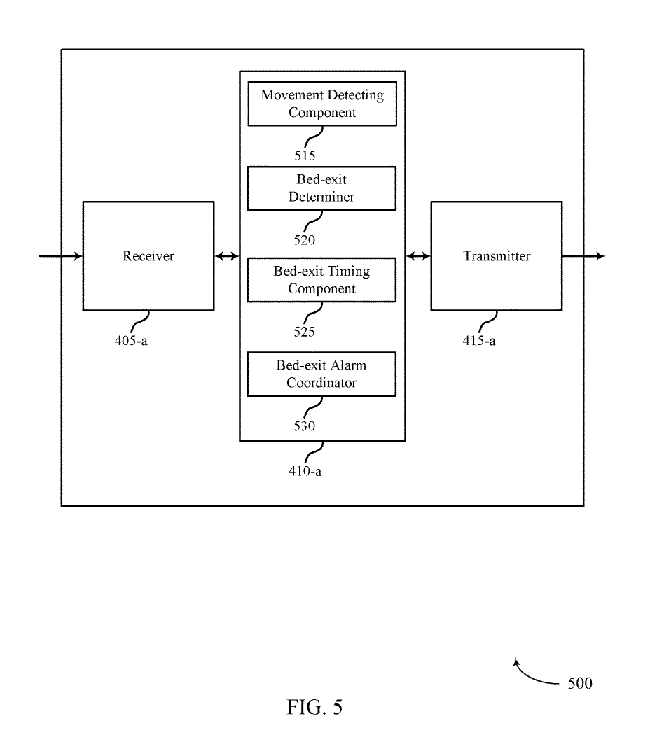

FIG. 5 shows a block diagram of a wireless device 500 that supports patient bed-exit prediction and detection in accordance with various aspects of the present disclosure. The wireless device 500 may be an example of aspects of a wireless device 400 or a central aggregator 205 described with reference to FIGS. 2-4. The wireless device 500 may include receiver 405-a, bed-exit detection manager 410-a, and transmitter 415-a. The wireless device 500 may also include a processor. Each of these components may be in communication with each other. The wireless device 500 may communicate via wired or wireless means.

The receiver 405-a may receive information (e.g., vital sign data, or movement data from sensor units such as pressure sensors and accelerometers) which may be passed on to other components of the device. The receiver 405-a may also perform the functions described with reference to the receiver 405 of FIG. 4. The transmitter 415-a may transmit signals received from other components of the wireless device 500. In some examples, the transmitter 415-a may be collocated with a receiver in a transceiver module. For example, the receive 405-a and transmitter 415-a may be an example of aspects of the transceiver 625 described with reference to FIG. 7. The transmitter 415-a may utilize a single antenna, or it may utilize a plurality of antennas.

The bed-exit detection manager 410-a may be an example of aspects of bed-exit detection manager 410 described with reference to FIG. 4. The bed-exit detection manager 410-a may include movement detecting component 515, bed-exit determiner 520, bed-exit timing component 525, and bed-exit alarm coordinator 530. The bed-exit detection manager 410-a may be an example of aspects of the bed-exit detection manager 410-b described with reference to FIG. 6. The bed-exit manager 410-a may receive, via receiver 405-a, pressure data from a pressure sensor disposed under a patient and accelerometry data from an accelerometer coupled with the patient. The pressure data and accelerometry data may be associated with movement of the patient.

The movement detecting component 515 may include circuitry, logic, hardware or software for detecting a pattern of movement of the patient. In some cases, the pattern of movement is detected using a combination of the pressure data and accelerometry data. In some embodiments, the movement detecting component 515 detects the pattern of movement from the pressure data and verifies the pattern of movement with the accelerometry data. In other embodiments, the movement detecting component 515 detects the pattern of movement from the accelerometry data and verifies the pattern of movement with the pressure data. Detecting the pattern of movement may include detecting the patient sitting up, rolling over, shifting, or repositioning one or more limbs.

The bed-exit determiner 520 may include circuitry, logic, hardware or software for determining whether the movement detected by the movement detecting component 515 is indicative of bed-exit. The determination may be based at least in part on the detected movement. In some cases, the bed-exit determiner 520 may make the determination by comparing the detected movement with a predetermined pattern of movement indicative of bed-exit. The determination may be based at least in part on a weighted algorithm to which the pressure data and the accelerometry data serve as inputs. The weighted algorithm may be specifically calibrate to the patient and may be dynamically updated. For example, the weighted algorithm may be updated based at least in part on the time of day, a duration of time since a previous bed-exit, or a frequency of previous bed-exits. In some cases, the weighted algorithm may be adjusted based on other information associated with the patient (e.g., information received from another sensor unit 110, a local computing device 115, a remote computing device 145, or input by a clinician).

The bed-exit timing component 525 may include circuitry, logic, hardware or software for determining when the detected bed-exit is going to occur or be attempted. For example the bed-exit timing component 525 may determine whether the detected movement is indicative of a bed-exit attempt occurring within a threshold time. In some cases, the threshold time may be less than a minute (e.g., the detected bed-exit may be a current bed-exit attempt). In some cases, the bed-exit timing component 525 may determine that the detected movement is indicative of a bed-exit attempt that will occur after the threshold time (e.g., after one minute). For example, the bed-exit timing component 525 may determine that the detected movement is associated with (e.g., predictive of) a future bed-exit attempt.

The bed-exit alarm coordinator 530 may include circuitry, logic, hardware or software for triggering (e.g., via transmitter 415-a) an alarm based at least in part on the determination that the detected movement is indicative of a bed-exit attempt. The bed-exit alarm coordinator 530 may convey the type of bed-exit (e.g., current or future) to the alarm. In some cases, the alarm is internal to bed-exit detection manager 410-a. In certain aspects, bed-exit alarm coordinator 530 may communicate with the alarm to adjust the sensitivity or level of the alarm. The adjustment may be based at least in part on the movement detected by the movement detecting component 515, or on the analysis of the bed-exit determiner 520.

FIG. 6 shows a diagram of a system 600 that supports patient bed-exit prediction and detection in accordance with various aspects of the present disclosure. System 600 may include a central aggregator 205-a, which may be an example of a central aggregator 205, wireless device 400, or wireless device 500 as described with reference to FIGS. 2, 4, and 5. System 600 may also include a pressure sensor 110-b-1 which may be an example of a pressure sensor 110-b described with reference to FIGS. 1-5. Pressure sensor 110-b-1 may be disposed under a patient that is lying in a medical bed and may be configured to collect pressure data associated with movement of the patient. System 600 may also include an accelerometer 110-a-1 which may be an example of an accelerometer 110-a described with reference to FIGS. 1-5. Accelerometer 110-a-1 may be coupled with the patient and may be configured to collect accelerometry data associated with movement of the patient. Central aggregator 205-a may be configured to determine whether the movement of the patient is indicative of bed-exit based at least in part on the collected pressure data and the collected accelerometry data. In some cases, the operations of central aggregator 205-a are performed by accelerometer 110-a-1.

Central aggregator 205-a may include bed-exit detection manager 410-b, which may be an example of a bed-exit detection manager 410 described with reference to FIGS. 4 and 5. Central aggregator 205-a may also include memory 610, processor 620, transceiver 625, and antenna 630. Each of these modules may communicate, directly or indirectly, with one another (e.g., via one or more buses). The memory 610 may be in electronic communication with the processor 620 and may include random access memory (RAM) and read only memory (ROM).

The memory 610 may store computer-readable, computer-executable software (e.g., software 615) including instructions that, when executed, cause the processor to perform various functions described herein (e.g., patient bed-exit prediction and detection, etc.). In some cases, the software 615 may not be directly executable by the processor but may cause a computer (e.g., when compiled and executed) to perform functions described herein.

The processor 620 may include an intelligent hardware device, (e.g., a central processing unit (CPU), a microcontroller, an application specific integrated circuit (ASIC), etc.). The transceiver 625 may communicate bi-directionally, via one or more antennas, wired, or wireless links, with one or more networks, as described above. The transceiver 625 may also include a modem to modulate the packets and provide the modulated packets to the antennas for transmission, and to demodulate packets received from the antennas. In some cases, central aggregator 205-a may include a single antenna 630. However, in some cases the device may have more than one antenna 630, which may be capable of concurrently transmitting or receiving multiple wireless transmissions.

FIG. 7 shows a flowchart illustrating a method 700 for patient bed-exit prediction and detection in accordance with various aspects of the present disclosure. The operations of method 700 may be implemented by a device such as a central aggregator 205, wireless device 400, wireless device 500, or its components as described with reference to FIGS. 2-5. For example, the operations of method 700 may be performed by the bed-exit detection manager 410 as described herein. In some examples, the central aggregator 205 may execute a set of codes to control the functional elements of the device to perform the functions described below. Additionally or alternatively, the central aggregator 205 may perform aspects the functions described below using special-purpose hardware.

At block 705, the method may include receiving pressure data from a pressure sensor disposed under a patient as described above with reference to FIGS. 2 and 3. In certain examples, the operations of block 705 may be performed by the movement detecting component 515 as described with reference to FIG. 5. At block 710, the method may include receiving accelerometry data from an accelerometer coupled with the patient as described above with reference to FIGS. 2 and 3. In certain examples, the operations of block 710 may be performed by the movement detecting component 515 as described with reference to FIG. 5.

At block 715, the method may include detecting movement of the patient based at least in part on the pressure data and the accelerometry data as described above with reference to FIGS. 2 and 3. In some cases, detecting movement includes detecting a pattern of movement of the patient from the pressure data and verifying the pattern of movement with the accelerometry data. In other cases, detecting movement includes detecting a pattern of movement of the patient from the accelerometry data and verifying the pattern of movement with the pressure data. In some embodiments, detecting includes detecting the patient sitting up, rolling over, shifting, or repositioning one or more limbs. In certain examples, the operations of block 715 may be performed by the movement detecting component 515 as described with reference to FIG. 5.

At block 720, the method may include determining whether the detected movement is indicative of bed-exit. The determination may be based at least in part on the detected movement as described above with reference to FIGS. 2 and 3. In some cases, determining includes comparing the detected movement with a predetermined pattern of movement indicative of bed-exit. In some embodiments, the determining is based at least in part on a weighted algorithm that is specifically calibrated to the patient. The pressure data and accelerometry data may be inputs to the weighted algorithm. The weighted algorithm may be dynamically updated based at least in part on the time of day, a duration of time since a previous bed-exit, or a frequency of previous bed-exits. In certain examples, the operations of block 720 may be performed by the bed-exit determiner 520 as described with reference to FIG. 5.

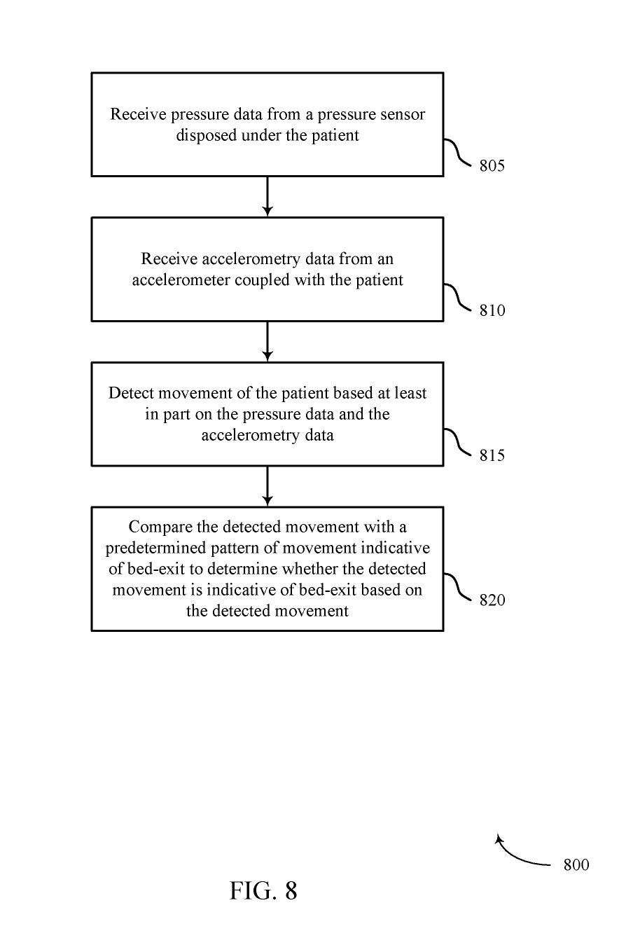

FIG. 8 shows a flowchart illustrating a method 800 for patient bed-exit prediction and detection in accordance with various aspects of the present disclosure. The operations of method 800 may be implemented by a device such as a central aggregator 205 or its components as described with reference to FIGS. 1 and 2. For example, the operations of method 800 may be performed by the bed-exit detection manager 410 as described herein. In some examples, the central aggregator 205 may execute a set of codes to control the functional elements of the device to perform the functions described below. Additionally or alternatively, the central aggregator 205 may perform aspects the functions described below using special-purpose hardware.

At block 805, the method may include receiving pressure data from a pressure sensor disposed under the patient as described above with reference to FIGS. 2 and 3. In certain examples, the operations of block 805 may be performed by the movement detecting component 515 as described with reference to FIG. 5. At block 810, the method may include receiving accelerometry data from an accelerometer coupled with the patient as described above with reference to FIGS. 2 and 3. In certain examples, the operations of block 810 may be performed by the movement detecting component 515 as described with reference to FIG. 5.

At block 815, the method may include detecting movement of the patient based at least in part on the pressure data and the accelerometry data as described above with reference to FIGS. 2 and 3. In certain examples, the operations of block 815 may be performed by the movement detecting component 515 as described with reference to FIG. 5. At block 820, the method may include comparing the detected movement with a predetermined movement indicative of bed-exit to determine whether the detected movement is indicative of bed-exit based at least in part on the detected movement as described above with reference to FIGS. 2 and 3. In certain examples, the operations of block 820 may be performed by the bed-exit determiner 520 as described with reference to FIG. 5.

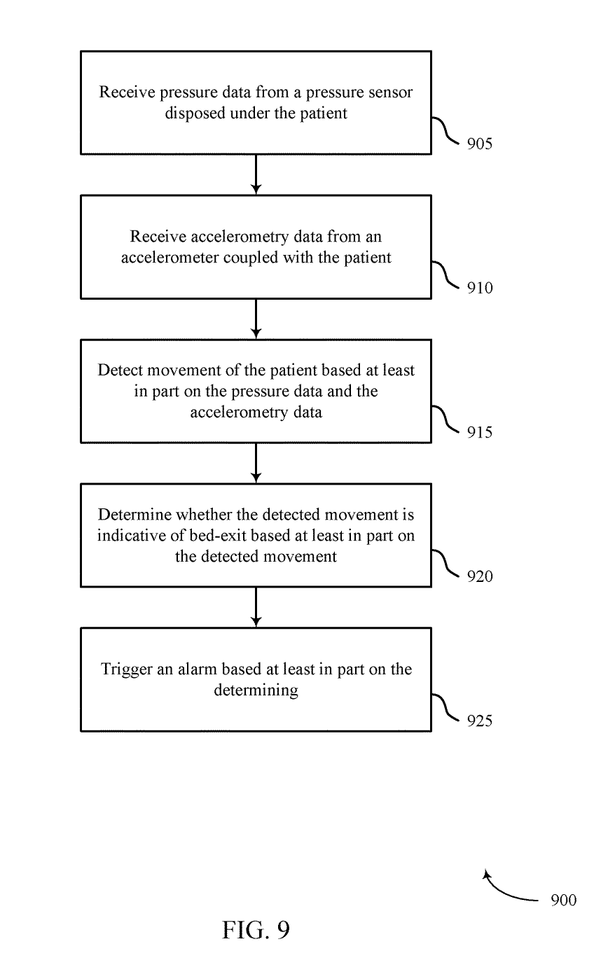

FIG. 9 shows a flowchart illustrating a method 900 for patient bed-exit prediction and detection in accordance with various aspects of the present disclosure. The operations of method 900 may be implemented by a device such as a central aggregator 205 or its components as described with reference to FIGS. 1 and 2. For example, the operations of method 900 may be performed by the bed-exit detection manager 410 as described herein. In some examples, the central aggregator 205 may execute a set of codes to control the functional elements of the device to perform the functions described below. Additionally or alternatively, the central aggregator 205 may perform aspects the functions described below using special-purpose hardware.

At block 905, the method may include receiving pressure data from a pressure sensor disposed under the patient as described above with reference to FIGS. 2 and 3. In certain examples, the operations of block 905 may be performed by the movement detecting component 515 as described with reference to FIG. 5. At block 910, the method may include receiving accelerometry data from an accelerometer coupled with the patient as described above with reference to FIGS. 2 and 3. In certain examples, the operations of block 910 may be performed by the movement detecting component 515 as described with reference to FIG. 5.

At block 915, the method may include detecting movement of the patient based at least in part on the pressure data and the accelerometry data as described above with reference to FIGS. 2 and 3. In certain examples, the operations of block 915 may be performed by the movement detecting component 515 as described with reference to FIG. 5. At block 920, the method may include determining whether the detected movement is indicative of bed-exit based at least in part on the detected movement as described above with reference to FIGS. 2 and 3. In some cases, the method includes determining whether the detected movement is indicative of a bed-exit attempt occurring within a threshold time. The threshold time may be less than a minute. In certain examples, the operations of block 920 may be performed by the bed-exit determiner 520 as described with reference to FIG. 5.

At block 925, the method may include triggering an alarm based at least in part on the determining as described above with reference to FIGS. 2 and 3. In some cases, triggering the alarm includes sending an indication of patient bed-exit to an alarm system. In some cases, the method includes adjusting the sensitivity of the alarm based at least in part on a condition of the patient, a medication level of the patient, or a previous fall history of the patient. In certain examples, the operations of block 925 may be performed by the alarm triggering component as described with reference to FIGS. 5 and 6.

It should be noted that these methods describe possible implementation, and that the operations and the steps may be rearranged or otherwise modified such that other implementations are possible. In some examples, aspects from two or more of the methods may be combined. For example, aspects of each of the methods may include steps or aspects of the other methods, or other steps or techniques described herein. Thus, aspects of the disclosure may provide for patient bed-exit prediction and detection.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed herein.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical (PHY) locations. Also, as used herein, including in the claims, “or” as used in a list of items (for example, a list of items prefaced by a phrase such as “at least one of” or “one or more”) indicates an inclusive list such that, for example, a list of at least one of A, B, or C means A or B or C or AB or AC or BC or ABC (i.e., A and B and C).

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

What is claimed is:

1. A method for predicting bed-exit of a patient, comprising:
receiving pressure data from a pressure sensor disposed under the patient;
receiving accelerometry data from an accelerometer coupled with the patient;
detecting movement of the patient based at least in part on the pressure data and the accelerometry data; and
determining whether the detected movement is indicative of bed-exit based at least in part on the detected movement.

2. The method of claim 1, wherein the determining comprises:
comparing the detected movement with a predetermined pattern of movement indicative of bed-exit.

3. The method of claim 1, wherein the determining is based at least in part on a weighted algorithm specifically calibrated to the patient, and wherein the pressure data and the accelerometry data are inputs to the weighted algorithm.

4. The method of claim 1, wherein the determining is based at least in part on a weighted algorithm that is dynamically updated based on the time of day, a duration of time since a previous bed-exit, a frequency of previous bed-exits, or a combination thereof, and wherein the pressure data and the accelerometry data are inputs to the weighted algorithm.

5. The method of claim 1, wherein the detecting comprises:
detecting a pattern of movement of the patient from the pressure data and verifying the pattern of movement with the accelerometry data.

6. The method of claim 1, wherein the detecting comprises:
detecting a pattern of movement of the patient from the accelerometry data and verifying the pattern of movement with the pressure data.

7. The method of claim 1, wherein the detecting comprises detecting the patient sitting up, rolling over, shifting, repositioning one or more limbs, or a combination thereof.

8. The method of claim 1, further comprising:
determining whether the detected movement is indicative of a bed-exit attempt occurring within a threshold time.

9. The method of claim 8, wherein the threshold time is less than a minute.

10. The method of claim 1, further comprising:
triggering an alarm based at least in part on the determining.

11. The method of claim 10, wherein a sensitivity of the alarm is adjusted based at least in part on a condition of the patient, a medication level of the patient, a previous fall history of the patient, or a combination thereof.

12. The method of claim 10, wherein triggering the alarm comprises:
sending an indication of patient bed-exit to an alarm system.

13. An apparatus for predicting bed-exit of a patient, comprising:
a processor;
memory in electronic communication with the processor; and
instructions stored in the memory and operable, when executed by the processor, to cause the apparatus to:
detect movement of the patient based at least in part on pressure data and accelerometry data associated with movement of the patient; and
determine whether the detected movement is indicative of bed-exit.

14. The apparatus of claim 13, wherein the instructions are operable to cause the apparatus to receive the pressure data and the accelerometry data wirelessly.

15. The apparatus of claim 13, wherein the instructions are operable to cause the apparatus to trigger an alarm indicative of bed-exit based at least in part on the detected movement.

16. A system for predicting bed-exit of a patient, comprising:
a pressure sensor configured to collect pressure data associated with movement of the patient;
an accelerometer configured to collect accelerometry data associated with movement of the patient; and
a central aggregator configured to determine whether the movement of the patient is indicative of bed-exit based at least in part on the collected pressure data and the collected accelerometry data.

17. The system of claim 16, wherein the pressure sensor is a bed mat pressure sensor.

18. The system of claim 16, wherein the accelerometer is coupled with the chest of the patient.

19. The system of claim 16, further comprising an alarm in communication with the central aggregator, the alarm configured to provide an indication of bed-exit.

20. The system of claim 16, wherein the central aggregator is configured to wirelessly communicate with the pressure sensor and the accelerometer.
